(19) United States
(12) Patent Application Publication
(10) Pub. No.: US 2023/0398137 A1
(43) Pub. Date: Dec. 14, 2023

(54) METHODS FOR LOWERING HBA1C LEVEL WITH A COMBINATION OF A BET BROMODOMAIN INHIBITOR AND A SODIUM DEPENDENT GLUCOSE TRANSPORT 2 INHIBITOR

(71) Applicant: Resverlogix Corp., Calgary (CA)

(72) Inventors: Kenneth Eugene LEBIODA, Calgary (CA); Christopher Ross Armstrong HALLIDAY, Calgary (CA); Aziz Naeem KHAN, Calgary (CA)

(21) Appl. No.: 18/032,876

(22) PCT Filed: Oct. 29, 2021
(86) PCT No.: PCT/IB2021/060045

§ 371 Date: Apr. 20, 2023

Related U.S. Application Data
(60) Provisional application No. 63/107,843, filed on Oct. 30, 2020.

(51) Int. Cl. A61K 31/7048; A61K 31/517; A61K 31/7034; A61P 3/08

(52) U.S. Cl. CPC A61K 31/7048; A61K 31/517; A61K 31/7034; A61P 3/08

(57) ABSTRACT

Described herein are methods for lowering glycated hemoglobin (HbA1c) level to treat and/or prevent a diabetes-related disease or disorder by administering to a subject in need thereof, a combination of a sodium-glucose transport protein 2 (SGLT2) inhibitor and a compound of Formula I or a stereoisomer, tautomer, pharmaceutically acceptable salt, or hydrate thereof, wherein the variables of Formula I are as defined herein.

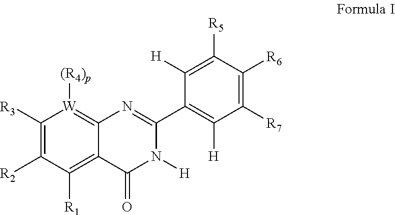

This application claims the benefit of priority to U.S. Provisional Application No. 63/107,843, filed Oct. 30, 2020, the contents of which are incorporated by reference herein in their entirety.

The present disclosure relates to methods for lowering glycated hemoglobin or hemoglobin A1c (HbA1c) level (i.e., blood HbA1c level) to treat and/or prevent a diabetes-related disease or disorder by administering to a subject in need thereof, a combination of a sodium-glucose transport protein 2 (SGLT2) inhibitor and a compound of Formula I or a stereoisomer, tautomer, pharmaceutically acceptable salt, or hydrate thereof.

HbA1c is the glycated or glucose-coated form of hemoglobin. Hemoglobin functions by transporting oxygen through the circulatory system and can become glycated or coated with glucose from the bloodstream. The HbA1c test is used to evaluate glucose control; it demonstrates an average of the blood sugar level over the past 90 days (the average lifespan of a red blood cell) and is expressed as a percentage (Sherwani et al. 2016). Testing HbA1c is recognized as a standard of care for monitoring diabetes, specifically type II diabetes (T2DM) (WHO 2011). The American Diabetes Association recommends testing HbA1c for diagnosing diabetes as an alternative to fasting plasma glucose of ≥7.0 mmol/L which was widely used prior to the standardization of the HbA1c diagnostic test (Khan et al. 2007). Nondiabetics usually fall within the 4.0%-5.6% HbA1c range, prediabetics usually have HbA1c levels of 5.7%-6.4%, while those with 6.5% or higher HbA1c levels have clinical diagnosed diabetes (American Diabetes Association 2011). Diabetes, and specially type II diabetes, is characterized by chronic elevated blood glucose levels (hyperglycemia) resulting from imbalanced hepatic glucose production and insulin secretion (Kharroubi and Darwish 2015).

Normalization of plasma glucose, as measured by HbA1c, in T2DM patients is known to be a target to improve insulin action, and to prevent the development of diabetic complications (Kharroubi and Darwish 2015).

Diabetes is known to be associated with several complications, such as neuropathy, nephropathy, retinopathy, and amputations, as well as comorbidities, including insulin-resistance (impaired glucose homeostasis), hyperglycemia, hyperinsulinemia, metabolic syndrome, progressive cognitive decline, elevated blood levels of fatty acids or glycerol, hyperlipidemia including hypertriglyceridemia, obesity, muscle quality decline, muscle atrophy, and sarcopenia (Fowler 2008; Vithian and Hurel 2010; Beckman and Creager 2016; Klein et al. 1984; Rangel et al. 2016; Zheng et al. 2019; Naqvi et al. 2017; Sheth et al. 2015; Bae et al. 2016; Yoon et al. 2016; Kalyani et al. 2015; Park et al. 2006; Hirata et al. 2019; Sugimoto et al. 2019; Ozturk et al. 2018).

Elevated levels of HbA1c have been correlated to decreases in cognitive function, defined as multiple mental abilities, including learning, thinking, reasoning, remembering, problem solving, decision making, and attention. The AgeCoDe cohort of 1,342 elderly individuals, which analyzed the association between HbA1c levels and the incidence of all-cause dementia and of Alzheimer's Disease dementia, observed that HbA1c levels of ≥6.5% were associated with a 2.8-fold increased risk of incident all-cause dementia and Alzheimer's Disease dementia. HbA1c levels of ≥7% were associated with an even greater risk of incident all-cause dementia and Alzheimer's Disease dementia, up to a 5-fold increased risk (Ramirez et al. 2014). An analysis of 8,888 Health and Retirement Study participants aged 50 years or older reported that diabetes was associated with a 10% faster rate of memory decline, with each 1% in HbA1c corresponding with a 0.05 SD decrease in memory score per decade (Marden et al. 2017). In a large observational study of 353,214 individuals with T2DM from the Swedish National Diabetes Registry, HbA1c levels of ≥10% increased the rate of dementia by 23% to 77% depending on a time-fixed or time-updated statistical analysis, concluding that lowering HbA1c and good general diabetes risk-factor control may help prevent dementia in T2DM patients (Rawshani et al. 2015). Similarly, another analysis of 378,299 people with T2DM and 1,886,022 age- and sex-matched controls identified in the Swedish National Diabetes Register found a linear association between HbA1c and the risk for Alzheimer's, vascular, and nonvascular dementia, citing poor glycemic control as a risk factor for developing dementia (Celis-Morales et al. 2020).

Individuals with T2DM are known to have a higher risk of cardiovascular mortality and morbidity than those without T2DM. Observational studies have reported an association between elevated HbA1c levels and cardiovascular risk in patients with T2DM. The UKPDS-35 evaluated 3,642 patients with newly diagnosed T2DM and illustrated that each 1% reduction in HbA1c was associated with 14%, 12%, and 16% reductions in the relative risk of myocardial infarction, stroke, and heart failure, respectively (Stratton et al. 2000). The EPIC-Norfolk study found that the cardiovascular risk and all-cause mortality of the participants (4,662 men and 5,570 women) had continuous associations with HbA1c levels, such that a 1% increase in HbA1c was associated with an relative-risk of death from any cause of 1.24 in men and 1.28 in women independent of age, body mass index, waist-to-hip ratio, systolic blood pressure, serum cholesterol concentration, cigarette smoking, and any history of cardiovascular diseases (Khaw et al. 2004). A meta-analysis of observational studies of associations between HbA1c and cardiovascular events in patients with T2DM illustrated relative risk estimate for coronary heart disease or stroke of 1.18 for each 1% increase in HbA1c (Selvin et al. 2004).

The observational evidence of an association between HbA1c levels and diabetes-associated complications and comorbidities, including those described above, is the basis for the guidelines that recommend the control of blood glucose, as measured by HbA1c, in patients with T2DM. Regulatory authorities (including the FDA and EMA) have approved medicines for the treatment of T2DM based on the use of HbA1c as the primary therapeutic endpoint (Shimazawa and Ikeda 2019).

SGLT2 inhibitors, which induce the secretion of glucose in the urine by inhibition of sodium glucose transport protein 2, have been shown to reduce HbA1c levels in patients with established cardiovascular disease, diabetes, and chronic kidney disease (Zinman et al. 2015; Neal et al. 2017; Perkovic et al. 2019; Wiviott et al. 2019). The ability of SGLT2 inhibitors to reduce elevated HbA1c levels in type 2 diabetes patients has been studied in several clinical trials, such as EMPA-REG OUTCOME for empaglifozin (NCT01131676); CANVAS Program for canaglifozin (NCT01032629 and NCT01989754); and DECLARE-TIMI 58 (NCT01730534) for dapaglifozin. To summarize, in the EMPA-REG OUTCOME trial, empaglifozin was shown to reduce HbA1c by 0.54% (95% CI, −0.58 to −0.49) in the 10-mg group and −0.60 percentage points (95% CI, −0.64 to −0.55) in the 25-mg group compared to placebo after 12 weeks (adjusted mean differences). By week 94, the adjusted mean differences in HbA1c levels between patients receiving empagliflozin and those receiving placebo were −0.42% (95% CI, −0.48 to −0.36) and −0.47% (95% CI, −0.54 to −0.41), respectively; and, at week 206, the differences were −0.24% (95% CI, −0.40 to −0.08) and −0.36% (95% CI, −0.51 to −0.20), respectively (Zinman et al. 2015). In the CANVAS Program, canaglifozin was also shown to have the ability to reduce elevated HbA1c levels, with the mean difference between the canagliflozin group and the placebo group being −0.58% (95% CI, −0.61 to −0.56) (p<0.001) over the duration of the trial (Neal et al. 2017). Similarly, in the CREDENCE trial of canagliflozin in patients with T2DM and albuminuric CKD, the least-squares mean level of HbA1c at 13 weeks was lower in the canagliflozin group than in the placebo group by 0.31% (95% CI, 0.26 to 0.37), and the between-group difference narrowed thereafter, with an overall mean difference in the reduction throughout the trial of 0.25% (95% CI, 0.20 to 0.31) (Perkovic et al. 2019). As for dapagliflozin, in the DECLARE-TIMI 58 study, patients in the dapagliflozin group had slightly lower HbA1c levels throughout the trial than patients in the placebo group, with an average least-squares mean absolute difference between the groups of 0.42% (95% CI, 0.40 to 0.45) (Wiviott et al. 2019).

No studies have been conducted on the efficacy of SGLT2 inhibitor therapies in patients with low HDL cholesterol (below 40 mg/dL for males and below 45 mg/dL for females) and a recent Acute Coronary Syndrome (ACS) (preceding 7-90 days) event. Thus, a significant unmet need still exists for the reduction of elevated HbA1c in patients with established cardiovascular disease and T2DM, as many patients still do not successfully reach their glycemic targets (i.e., HbA1c levels below 7.0%) with SGLT2 therapies (Owen et al 2017).

Apabetalone (RVX-208 or RVX000222) is a first-in-class Bromodomain and Extra-Terminal (BET)-inhibitor (BETi) that binds selectively to the second bromodomain of BET proteins. BET proteins (BRD2, BRD3, BRD4, and BRDT) are epigenetic readers that recognize and bind to acetylated lysines on histones 3 and 4 and on some transcription factors. Histone bound BETs recruit transcription factors and machinery to gene enhancer and promoter sites, facilitating the transcription of proximal genes. Chronic disease profoundly alters the acetylation landscape (Chen et al. 2005; Villagra et al. 2010; Bayarsaihan 2011), relocating BET proteins to the super-enhancers and promoters of genes involved in inflammation, lipid metabolism, and vascular function (Huang et al. 2009; Brown et al. 2014; Das et al. 2017). Apabetalone prevents BET protein translocation, inhibiting the transcription of genes that drive chronic diseases. Apabetalone treatment, by targeting BET proteins, is characterized by multipronged effects which are augmented in conditions with more pronounced maladaptive BET regulation.

A recently completed clinical Phase 3 trial (BETonMACE; NCT02586155) evaluated the effect on Major Adverse Cardiac events (MACE) of RVX-208 in T2DM patients who differ from the patient populations in the SGLT2 trials discussed above in that the T2DM patients in BETonMACE had low HDL cholesterol (below 40 mg/dL for males and below 45 mg/dL for females) and a recent Acute Coronary Syndrome (ACS) (preceding 7-90 days) event. Moreover, all patients in BETonMACE received high intensity or maximum tolerated statin treatment. BETonMACE was the first clinical trial to chronically dose high-risk cardiovascular disease patients with T2DM with the combination of a BET inhibitor and an SGLT2 inhibitor.

In the same BETonMACE clinical trial, the effects of RVX-208 monotherapy, SGLT2 inhibitor monotherapy, and the RVX-208 and SGLT2 inhibitor combination therapy on HbA1c levels in T2DM patients with a recent ACS were also evaluated. Significantly, RVX-208 monotherapy did not demonstrate the ability to statistically reduce levels of HbA1c in T2DM patients with recent ACS in the recently completed phase 3 BETonMACE trial (Ray et al. 2020). As indicated by the data and results presented in this application, monotherapy with an SGLT2 inhibitor also did not demonstrate a statistically significant ability to reduce levels of HbA1c in T2DM patients with a recent ACS.

Surprisingly, as detailed in Example 2, we found that patients treated with the combination of RVX-208 and an SGLT2 inhibitor showed pronounced reduction of HbA1c, compared to treatment with either therapy alone. The summary of the results discussed below and the detailed description of the results in Example 2 demonstrate that RVX-208 or SGLT2 inhibitors by themselves did not reduce HbA1c in patients with recent ACS and T2DM. However, when apabetalone was combined with a SGLT2 inhibitor, an unexpected and statistically significant reduction of HbA1c was observed.

Of note, RVX-208 in combination with SGLT2 inhibitors decreased HbA1c from a median of 8.2% at baseline to a median of 7.8% at the last visit on treatment (LVT). This level of HbA1c reduction was unexpected because, as mentioned above, the patients in BETonMACE were receiving maximum tolerated statin therapy, and statin has been shown to significantly increase Hb1Ac levels and worsen glycemic control in both diabetic and non-diabetic patients (Ooba et al. 2016; Cui et al, 2018). Therefore, it was unexpected that the HbA1c reductions reported in the SGLT2 clinical trials discussed above would also be observed in the BETonMACE patient population when combined with treatment with a compound of Formula I. Indeed, and comparatively, the SGLT2 inhibitor monotherapy among the BETonMACE patients had a median HbA1c of 8.0% at baseline and a median HbA1c of 8.2% at LVT (i.e., no reduction in median HbA1c). The RVX-208 monotherapy group had a median HbA1c of 7.3% at baseline and a median HbA1c of 7.3% at LVT (i.e., also no reduction in median HbA1c). Thus, the reduction in HbA1c observed when a compound of Formula I is administered with an SGLT2 inhibitor exceeded the additive effects of apabetalone and the SGLT2 inhibitor individually.

Accordingly, the technical solution provided by the present disclosure includes methods for lowering HbA1c level to treat and/or prevent a diabetes-related disease or disorder as defined herein by administering to a subject in need thereof, a combination of a sodium-glucose transport protein 2 (SGLT2) inhibitor and a compound of Formula I or Ia or a stereoisomer, tautomer, pharmaceutically acceptable salt, or hydrate thereof.

Compounds of Formula I have previously been described in U.S. Pat. No. 8,053,440, which is incorporated herein by reference. Compounds of Formula I include:

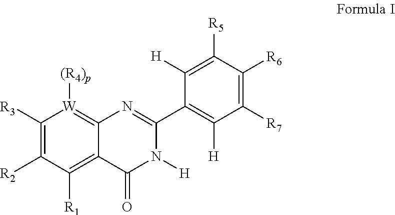

stereoisomers, tautomers, pharmaceutically acceptable salts, or hydrates thereof,
wherein:
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₅ and R₇ are each independently selected from alkyl, alkoxy, amino, halogen, and hydrogen;
R₆ is selected from amino, amide, alkyl, hydrogen, hydroxyl, piperazinyl, and alkoxy;
W is selected from C and N, wherein:
   if W is N, then p is 0 or 1, and
   if W is C, then p is 1; and
for W—(R₄)ₚ, W is C, p is 1 and R₄ is H, or W is N and p is 0.

Apabetalone (RVX-208 or RVX000222) is a representative example of Formula I.

In some embodiments, the diabetes-related disease or disorder treated and/or prevented by a method of the disclosure is a diabetic comorbidity associated with elevated HbA1c levels (e.g., ≥6.5%+10%). Non-limiting examples of such comorbidities are insulin resistance (impaired glucose homeostasis), hyperglycemia, hyperinsulinemia, metabolic syndrome, progressive cognitive decline, elevated blood levels of fatty acids or glycerol, hyperlipidemia including hypertriglyceridemia, obesity, muscle quality decline, muscle atrophy, sarcopenia, and a combination thereof.

In some embodiments, the diabetes-related disease or disorder treated and/or prevented by a method of the disclosure is a complication of diabetes associated with elevated HbA1c levels. Non-limiting examples of such a complication of diabetes include neuropathy, nephropathy, retinopathy, amputations, and a combination thereof.

In some embodiments, the diabetes-related disease or disorder treated and/or prevented by a method of the disclosure is another diabetic comorbidity associated with elevated HbA1c levels, namely dementia associated with elevated HbA1c levels. Non-limiting examples of dementia associated with elevated HbA1c levels include mild cognitive impairment, vascular dementia, Alzheimer's disease dementia, Lewy body dementia, frontotemporal dementia, mixed dementia (vascular dementia and Alzheimer's disease), and a combination thereof.

In some embodiments, the compound of Formula I or Ia or a stereoisomer, tautomer, pharmaceutically acceptable salt, or hydrate thereof is administered simultaneously with a SGLT2 inhibitor. In some embodiments, the compound of Formula I or Ia or a stereoisomer, tautomer, pharmaceutically acceptable salt, or hydrate thereof is administered sequentially with the SGLT2 inhibitor. In some embodiments, the compound of Formula I or Ia or a stereoisomer, tautomer, pharmaceutically acceptable salt, or hydrate thereof is administered in a single pharmaceutical composition with the SGLT2 inhibitor. In some embodiments, the compound of Formula I or Ia or a stereoisomer, tautomer, pharmaceutically acceptable salt, or hydrate thereof and the SGLT2 inhibitor are administered as separate compositions.

In some embodiments, the compound of Formula Ia is selected from

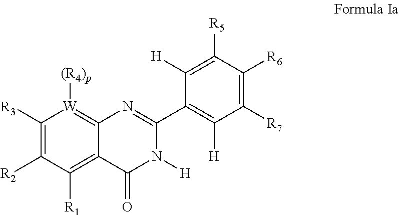

or a stereoisomer, tautomer, pharmaceutically acceptable salt, or hydrate thereof,
wherein:
R₁ and R₃ are each independently selected from alkoxy, alkyl, and hydrogen;
R₂ is selected from alkoxy, alkyl, and hydrogen;
R₅ and R₇ are each independently selected from alkyl, alkoxy, and hydrogen;
R₆ is selected from alkyl, hydroxyl, and alkoxy;
W is selected from C and N, wherein:
   if W is N, then p is 0 or 1, and
   if W is C, then p is 1; and
for W—(R₄)ₚ, W is C, p is 1 and R₄ is H, or W is N and p is 0.

In some embodiments, the compound of Formula I or Ia is 2-(4-(2-hydroxyethoxy)-3,5-dimethylphenyl)-5,7-dimethoxyquinazolin-4(3H)-one (RVX-208; RVX000222) or a pharmaceutically acceptable salt thereof.

In some embodiments, the dose of 2-(4-(2-hydroxyethoxy)-3,5-dimethylphenyl)-5,7-dimethoxyquinazolin-4(3H)-one is between 100-300 mg per day.

In some embodiments, the compound of Formula I is given once a day. In some embodiments, it is given twice a day.

In some embodiments, the SGLT2 inhibitor is empagliflozin, canagliflozin, dapagliflozin, remogliflozin, ipragliflozin, bexagliflozin, ertugliflozin, sotagliflozin, luseogliflozin, tofogliflozin, or HM41322.

In some embodiments, the SGLT2 inhibitor is empagliflozin, canagliflozin, or dapagliflozin.

In some embodiments, the SGLT2 inhibitor is dapagliflozin.

In some embodiments, the dose of dapagliflozin is between 5-10 mg

In some embodiments, the dose of dapagliflozin is 5 mg or 10 mg.

In some embodiments, the disclosure provides methods for lowering HbA1c level to treat and/or prevent a diabetes-related disease or disorder that is a diabetic comorbidity associated with elevated HbA1c levels selected from insulin resistance (impaired glucose homeostasis), hyperglycemia, hyperinsulinemia, metabolic syndrome, and a combination thereof.

In some embodiments, the disclosure provides methods for lowering HbA1c level to treat and/or prevent a diabetes-related disease or disorder that is a diabetic comorbidity associated with elevated HbA1c levels that is insulin resistance (impaired glucose homeostasis).

In some embodiments, the disclosure provides methods for lowering HbA1c level to treat and/or prevent a diabetes-related disease or disorder that is a diabetic comorbidity associated with elevated HbA1c levels selected from hyperglycemia, hyperinsulinemia, and a combination thereof.

In some embodiments, the disclosure provides methods for lowering HbA1c level to treat and/or prevent a diabetes-related disease or disorder that is a complication of diabetes associated with elevated HbA1c levels selected from neuropathy, nephropathy, retinopathy, and a combination thereof. In some embodiments, the disclosure provides methods for treating for lowering HbA1c level to treat and/or prevent a diabetes-related disease or disorder that is a diabetic comorbidity associated with elevated HbA1c levels that is dementia associated with elevated HbA1c levels selected from mild cognitive impairment, vascular dementia, Alzheimer's disease dementia, Lewy body dementia, frontotemporal dementia, mixed dementia (vascular dementia and Alzheimer's disease), and a combination thereof.

### BRIEF DESCRIPTION OF THE DRAWINGS

FIG. 1 depicts a comparison of the median change of HbA1c from baseline to LVT in patients administered RVX-208 with SGLT2 inhibitors versus patients administered placebo with SGLT2 inhibitors.

FIG. 2 depicts a comparison of the median change of HbA1c from baseline to LVT of HbA1c in patients administered RVX-208 with SGLT2 inhibitors versus patients administered RVX-208 without SGLT2 inhibitors.

FIG. 3 depicts a drug interaction matrix, comparing the median change from baseline to LVT of HbA1c in patients administered RVX-208 with or without SGLT2 inhibitors.

FIG. 4 depicts a drug interaction matrix, comparing the mean change from baseline to LVT of HbA1c in patients administered RVX-208 with or without SGLT2 inhibitors.

### DEFINITIONS

By “optional” or “optionally” it is meant that the subsequently described event or circumstance may or may not occur, and that the description includes instances where the event or circumstance occurs and instances in which is does not. For example, “optionally substituted aryl” encompasses both “aryl” and “substituted aryl” as defined below. It will be understood by those skilled in the art, with respect to any group containing one or more substituents, that such groups are not intended to introduce any substitution or substitution patterns that are sterically impractical, synthetically non-feasible, and/or inherently unstable.

As used herein, the term “hydrate” refers to a crystal form with either a stoichiometric or non-stoichiometric amount of water incorporated into the crystal structure.

The term “alkenyl” as used herein refers to an unsaturated straight or branched hydrocarbon having at least one carbon-carbon double bond, such as a straight or branched group of 2-8 carbon atoms, referred to herein as (C₂-C₈) alkenyl. Exemplary alkenyl groups include, but are not limited to, vinyl, allyl, butenyl, pentenyl, hexenyl, butadienyl, pentadienyl, hexadienyl, 2-ethylhexenyl, 2 propyl 2-butenyl, and 4-(2-methyl-3-butene)-pentenyl.

The term “alkoxy” as used herein refers to an alkyl group attached to an oxygen (O-alkyl). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, an alkyl, alkenyl or alkynyl group of 1-8 carbon atoms, referred to herein as (C₁-C₈) alkoxy. Exemplary alkoxy groups include, but are not limited to, methoxy and ethoxy.

The term “alkyl” as used herein refers to a saturated straight or branched hydrocarbon, such as a straight or branched group of 1-8 carbon atoms, referred to herein as (C₁-C₈) alkyl. Exemplary alkyl groups include, but are not limited to, methyl, ethyl, propyl, isopropyl, 2-methyl-1-propyl, 2-methyl-2-propyl, 2-methyl-1-butyl, 3 methyl-1-butyl, 2-methyl-3-butyl, 2,2-dimethyl-1-propyl, 2-methyl-1-pentyl, 3 methyl-1-pentyl, 4-methyl-1-pentyl, 2-methyl-2-pentyl, 3-methyl-2-pentyl, 4 methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “amide” as used herein refers to the form NRₐC(O)(R_{b}) or C(O)NR_{b}R_{c}, wherein Rₐ, R_{b}, and R_{c} are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, R_{b}, or R_{c}. The amide also may be cyclic, for example R_{b} and R_{c}, may be joined to form a 3- to 8-membered ring, such as 5- or 6-membered ring. The term “amide” encompasses groups such as sulfonamide, urea, ureido, carbamate, carbamic acid, and cyclic versions thereof. The term “amide” also encompasses an amide group attached to a carboxy group, e.g., amide-COOH or salts such as amide-COONa, an amino group attached to a carboxy group (e.g., amino-COOH or salts such as amino-COONa).

The term “amine” or “amino” as used herein refers to the form NR_{d}Rₑ or N(R_{d})Rₑ, where R_{d} and Rₑ are independently selected from alkyl, alkenyl, alkynyl, aryl, arylalkyl, carbamate, cycloalkyl, haloalkyl, heteroaryl, heterocycle, and hydrogen. The amino can be attached to the parent molecular group through the nitrogen. The amino also may be cyclic, for example, any two of R_{d} and Rₑ may be joined together or with the N to form a 3- to 12-membered ring (e.g., morpholino or piperidinyl). The term amino also includes the corresponding quaternary ammonium salt of any amino group. Exemplary amino groups include, but are not limited to, alkylamino groups, wherein at least one of R_{d} and Rₑ is an alkyl group. In some embodiments, R_{d} and Rₑ each may be optionally substituted with hydroxyl, halogen, alkoxy, ester, or amino.

The term “aryl” as used herein refers to a mono-, bi-, or other multi carbocyclic, aromatic ring system. The aryl group can optionally be fused to one or more rings selected from aryls, cycloalkyls, and heterocyclyls. The aryl groups of the present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms, referred to herein as “(C₆) aryl.”

The term “arylalkyl” as used herein refers to an alkyl group having at least one aryl substituent (e.g., aryl-alkyl). Exemplary arylalkyl groups include, but are not limited to, arylalkyls having a monocyclic aromatic ring system, wherein the ring comprises 6 carbon atoms, referred to herein as “(C₆) arylalkyl.”

The term “carbamate” as used herein refers to the form R_{g}OC(O)N(Rₕ), R_{g}OC(O)N(Rₕ)Rᵢ, or OC(O)NRₕRᵢ, wherein R_{g}, Rₕ, and Rᵢ are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (e.g., wherein at least one of R_{g}, Rₕ and Rᵢ are independently selected from aryl and heteroaryl, such as pyridine, pyridazine, pyrimidine, and pyrazine).

The term “carbocycle” as used herein refers to an aryl or cycloalkyl group.

The term “carboxy” as used herein refers to COOH or its corresponding carboxylate salts (e.g., COONa). The term carboxy also includes “carboxycarbonyl,” e.g., a carboxy group attached to a carbonyl group, e.g., C(O)—COOH or salts, such as C(O)—COONa.

The term “cycloalkoxy” as used herein refers to a cycloalkyl group attached to an oxygen.

The term “cycloalkyl” as used herein refers to a saturated or unsaturated cyclic, bicyclic, or bridged bicyclic hydrocarbon group of 3-12 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl saturated or unsaturated, aryl, or heterocyclyl groups.

The term “dicarboxylic acid” as used herein refers to a group containing at least two carboxylic acid groups such as saturated and unsaturated hydrocarbon dicarboxylic acids and salts thereof. Exemplary dicarboxylic acids include, but are not limited to, alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide, and thioketone. Dicarboxylic acids include, but are not limited to, succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides (for example, succinic anhydride and succinimide).

The term “ester” refers to the structure C(O)O—, C(O)ORⱼ, RₖC(O)O—Rⱼ, or RₖC(O)O—, where O is not bound to hydrogen, and Rⱼ and Rₖ can independently be selected from alkoxy, aryloxy, alkyl, alkenyl, alkynyl, amide, amino, aryl, arylalkyl, cycloalkyl, ether, haloalkyl, heteroaryl, and heterocyclyl. Rₖ can be a hydrogen, but Rⱼ cannot be hydrogen. The ester may be cyclic, for example the carbon atom and the oxygen atom and Rₖ, or Rⱼ and Rₖ may be joined to form a 3- to 12-membered ring. Exemplary esters include, but are not limited to, alkyl esters wherein at least one of Rⱼ and Rₖ is alkyl, such as O—C(O) alkyl, C(O)—O-alkyl, and alkyl C(O)—O-alkyl. Exemplary esters also include aryl or heteoaryl esters, e.g., wherein at least one of Rⱼ and Rₖ is a heteroaryl group such as pyridine, pyridazine, pyrimidine, and pyrazine, such as a nicotinate ester. Exemplary esters also include reverse esters having the structure RₖC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The terms “halo” or “halogen” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example, 1 to 3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C₂-C₅) heteroaryl.”

The terms “heterocycle,” “heterocyclyl,” or “heterocyclic” as used herein refer to a saturated or unsaturated 3, 4, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include, but are not limited to, acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure C(O)—Rₙ (such as acetyl, C(O)CH₃) or Rₙ—C(O)—Rₒ. The ketone can be attached to another group through Rₙ or Rₒ. Rₙ and Rₒ can be alkyl, alkenyl, alkynyl, cycloalkyl, heterocyclyl or aryl, or Rₙ and Rₒ can be joined to form a 3- to 12 membered ring.

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide, and thioketone.

The term “thioalkyl” as used herein refers to an alkyl group attached to a sulfur (S-alkyl).

“Alkyl,” “alkenyl,” “alkynyl”, “alkoxy”, “amino”, and “amide” groups can be optionally substituted with or interrupted by or branched with at least one group selected from alkoxy, aryloxy, alkyl, alkenyl, alkynyl, amide, amino, aryl, arylalkyl, carbamate, carbonyl, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide, thioketone, ureido, and N. The substituents may be branched to form a substituted or unsubstituted heterocycle or cycloalkyl.

As used herein, a suitable substitution on an optionally substituted substituent refers to a group that does not nullify the synthetic or pharmaceutical utility of the compounds of the present disclosure or the intermediates useful for preparing them. Examples of suitable substitutions include, but are not limited to: C₁-C₈ alkyl, C₂-C₈ alkenyl or alkynyl; C₆ aryl, 5- or 6-membered heteroaryl; C₃-C₇ cycloalkyl; C₁-C₈ alkoxy; C₆ aryloxy; CN; OH; oxo; halo, carboxy; amino, such as NH(C₁-C₈ alkyl), N(C₁-C₈ alkyl)₂, NH((C₆)aryl), or N((C₆)aryl)₂; formyl; ketones, such as CO(C₁-C₈ alkyl), —CO((C₆ aryl) esters, such as CO₂(C₁-C₈ alkyl) and CO₂(C₆ aryl). One of skill in art can readily choose a suitable substitution based on the stability and pharmacological and synthetic activity of the compound of the present disclosure.

The term “pharmaceutically acceptable composition” as used herein refers to a composition comprising at least one compound as disclosed herein formulated together with one or more pharmaceutically acceptable carriers.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of Formula I. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to, sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature, are capable of forming base salts with various pharmacologically acceptable cations. Examples of such salts include, but are not limited to, alkali metal or alkaline earth metal salts and, particularly, calcium, magnesium, sodium, lithium, zinc, potassium, and iron salts.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

The compounds of Formula I or Ia may contain one or more chiral centers and/or double bonds and, therefore, exist as stereoisomers, such as geometric isomers, enantiomers, or diastereomers. The term “stereoisomers” when used herein consists of all geometric isomers, enantiomers, or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds for use in the methods of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of Formula I or Ia. The present disclosure encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration, wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

The compounds of Formula I disclosed herein may exist as tautomers and both tautomeric forms are intended to be encompassed by the scope of the present disclosure, even though only one tautomeric structure is depicted.

As used herein, the term “SGLT2 inhibitor” refers a substance, such as a small molecule organic chemistry compound (≤1 kDa) or a large biomolecule such as a peptide (e.g., a soluble peptide), protein (e.g., an antibody), nucleic acid (e.g., siRNA) or a conjugate combining any two or more of the foregoing, that possesses the activity of inhibiting sodium-glucose transport protein 2 (SGLT2). Non-limiting examples of SGLT2 inhibitors include empagliflozin, canagliflozin, dapagliflozin, remogliflozin, ipragliflozin, HM41322, bexagliflozin, ertugliflozin, sotagliflozin, luseogliflozin, tofogliflozin, or a pharmaceutically acceptable salt of any of the foregoing. Additional examples of SGLT2 inhibitors are disclosed in WO01/027128, WO04/013118, WO04/080990, EP1852439A1, WO01/27128, WO03/099836, WO2005/092877, WO2006/034489, WO2006/064033, WO2006/117359, WO2006/117360, WO2007/025943, WO2007/028814, WO2007/031 548, WO2007/093610, WO2007/128749, WO2008/049923, WO2008/055870, and WO2008/055940, each of which is incorporated herein by reference in its entirety.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to reducing the progression of a disease or disorder, either physically, e.g., stabilization of a discernible symptom, physiologically, e.g., stabilization of a physical parameter, or both. In yet another embodiment, “treatment” or “treating” refers to delaying the onset or progression of a disease or disorder. For example, treating a cholesterol disorder may comprise decreasing blood cholesterol levels.

As used herein, “prevention” or “preventing” refers to a reduction of the risk of acquiring a given disease or disorder or a symptom of a given disease or disorder.

As used herein, “dementia associated with elevated HbA1c levels” refers to dementia, such as mild cognitive impairment, vascular dementia, Alzheimer's disease dementia, Lewy body dementia, frontotemporal dementia, and mixed dementia (vascular dementia and Alzheimer's disease), and a combination thereof, whereby a subject suffering therefrom has a HbA1c level in blood of ≥6.5%+10% (i.e., 6.5%-7.15%), if or when measured.

As used herein, “progression of cognitive decline” refers to the self-reported experience of worsening or more frequent confusion or memory loss, whereby a subject suffering therefrom has a HbA1c level in blood of ≥6.5%+10%, if or when measured. It is a form of cognitive impairment and one of the earliest noticeable symptoms of Alzheimer's disease and disease-related dementias.

As used herein, “mild cognitive impairment (MCI)” refers to the stage between the expected cognitive decline of normal aging and the more serious decline of dementia, whereby a subject suffering therefrom has a HbA1c level in blood of ≥6.5%+10%, if or when measured. MCI may increase risk of later developing dementia caused by Alzheimer's disease or other neurological conditions and thus is a potential early indicator of more serious disease-related dementia.

As used herein, “diabetes-related disease or disorder” refers to diseases, disorders and conditions that are complications of diabetes and/or comorbidities of diabetes associated with elevated HbA1c levels, whereby a subject suffering therefrom has a HbA1c level in blood of ≥6.5%+10%, if or when measured. Non-limiting examples of a diabetes-related disease or disorder that is a complication of diabetes or diabetic complication associated with elevated HbA1c levels are neuropathy, nephropathy, retinopathy, amputations, and a combination thereof. Non-limiting examples of a diabetes-related disease or disorder that is a comorbidity of diabetes or diabetic comorbidity associated with elevated HbA1c levels are insulin resistance (impaired glucose homeostasis), hyperglycemia, hyperinsulinemia, metabolic syndrome, progressive cognitive decline, elevated blood levels of fatty acids or glycerol, hyperlipidemia including hypertriglyceridemia, obesity, muscle quality decline, muscle atrophy, sarcopenia, and a combination thereof. Another non-limiting example of a diabetes-related disease or disorder that is a comorbidity of diabetes or diabetic comorbidity associated with elevated HbA1c levels is dementia associated with elevated HbA1c levels.

### EXEMPLARY EMBODIMENTS

In one embodiment, the present disclosure provides a method for lowering glycated hemoglobin (HbA1c) level to treat and/or prevent a diabetes-related disease or disorder, or a method for treating and/or preventing a diabetes-related disease or disorder by lowering glycated hemoglobin (HbA1c) level, the method comprising administering to a subject in need thereof, a combination of a sodium-glucose transport protein 2 (SGLT2) inhibitor and a compound of Formula I:

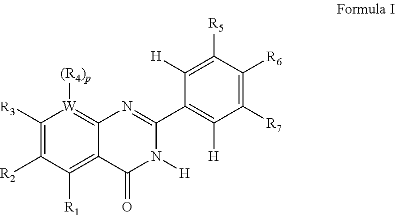

or a stereoisomer, tautomer, pharmaceutically acceptable salt, or hydrate thereof,
wherein:
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₅ and R₇ are each independently selected from alkyl, alkoxy, amino, halogen, and hydrogen;
R₆ is selected from amino, amide, alkyl, hydrogen, hydroxyl, piperazinyl, and alkoxy;
W is selected from C and N, wherein:
   if W is N, then p is 0 or 1, and
   if W is C, then p is 1; and
for W—(R₄)ₚ, W is C, p is 1 and R₄ is H, or W is N and p is 0.

In one embodiment, the compound of Formula I is a compound of Formula Ia:

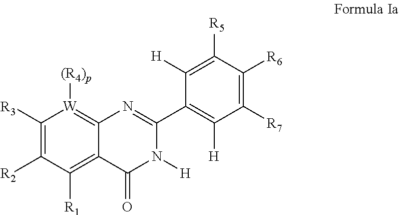

or a stereoisomer, tautomer, pharmaceutically acceptable salt, or hydrate thereof,
wherein:

R₁ and R₃ are each independently selected from alkoxy, alkyl, and hydrogen;
R₂ is selected from alkoxy, alkyl, and hydrogen;
R₅ and R₇ are each independently selected from alkyl, alkoxy, amino, halogen, and hydrogen;
R₆ is selected from alkyl, hydroxyl, and alkoxy;
W is selected from C and N, wherein:
   if W is N, then p is 0 or 1, and
   if W is C, then p is 1; and
for W—(R₄)ₚ, W is C, p is 1 and R₄ is H, or W is N and p is 0.

In one embodiment, the compound of Formula I or Ia is 2-(4-(2-hydroxyethoxy)-3,5-dimethylphenyl)-5,7-dimethoxyquinazolin-4(3H)-one (RVX-208 or RVX000222) or a pharmaceutically acceptable salt thereof.

In one embodiment, the method of the disclosure comprises administering to the subject, a daily dose of 100-300 mg of 2-(4-(2-hydroxyethoxy)-3,5-dimethylphenyl)-5,7-dimethoxyquinazolin-4(3H)-one or an equivalent amount of a pharmaceutically acceptable salt thereof.

In one embodiment, the method of the disclosure comprises administering to the subject, a daily dose of 200 mg of 2-(4-(2-hydroxyethoxy)-3,5-dimethylphenyl)-5,7-dimethoxyquinazolin-4(3H)-one or an equivalent amount of a pharmaceutically acceptable salt thereof.

In one embodiment, the SGLT2 inhibitor is selected from empagliflozin, canagliflozin, dapagliflozin, bexagliflozin, ertugliflozin, sotagliflozin, luseogliflozin, tofogliflozin, and HM41322.

In one embodiment, the SGLT2 inhibitor is selected from empagliflozin, canagliflozin, and dapagliflozin.

In one embodiment, the compound of Formula I or Ia is administered simultaneously with the SGLT2 inhibitor as separate compositions.

In one embodiment, the compound of Formula I or Ia is administered with the SGLT2 inhibitor as a single composition.

In one embodiment, the subject is a human.

In one embodiment, the subject is a human on statin therapy. In one embodiment, the subject is a human on high intensity or maximum tolerated statin therapy. In one embodiment, the high intensity statin treatment or therapy refers to a daily dose of at least 20 mg, or at least 40 mg, or 20-80 mg, or 20-40 mg, or 40-80 mg. In one embodiment, the maximum tolerated statin treatment or therapy refers to a daily dose of at least 40 mg, or 40 mg-80 mg, or 80 mg. In one embodiment, the subject is on rosuvastatin therapy. In one embodiment, the subject is on atorvastatin therapy.

In one embodiment, the subject is a human with type 2 diabetes and low HDL cholesterol (below 40 mg/dL for males and below 45 mg/dL for females) and a recent acute coronary syndrome (ACS) (preceding 7-90 days).

In one embodiment, the diabetes-related disease or disorder is a diabetic comorbidity associated with elevated HbA1c levels. In one embodiment, the diabetes-related disease or disorder is a diabetic comorbidity associated with elevated HbA1c levels that is insulin resistance (impaired glucose homeostasis). In one embodiment, the diabetes-related disease or disorder is a diabetic comorbidity associated with elevated HbA1c levels that is selected from muscle quality decline, muscle atrophy, sarcopenia, and a combination thereof. In one embodiment, the diabetes-related disease or disorder is a diabetic comorbidity associated with elevated HbA1c levels that is dementia associated with elevated HbA1c levels. In one embodiment, the dementia associated with elevated HbA1c levels is selected from mild cognitive impairment, vascular dementia, Alzheimer's disease dementia, Lewy body dementia, frontotemporal dementia, mixed dementia (vascular dementia and Alzheimer's disease), and a combination thereof.

In one embodiment, the diabetes-related disease or disorder is a complication of diabetes associated with elevated HbA1c levels. In one embodiment, the complication of diabetes is selected from nephropathy, neuropathy, retinopathy, and a combination thereof.

In one embodiment, the present disclosure provides a method for lowering glycated hemoglobin (HbA1c) level, the method comprising administering to a subject in need thereof, a combination of a sodium-glucose transport protein 2 (SGLT2) inhibitor and a compound of Formula I or Formula Ia or a stereoisomer, tautomer, pharmaceutically acceptable salt, or hydrate thereof as defined above. In one embodiment, the method for lowering HbA1c level treats and/or prevents a diabetes-related disease or disorder. Exemplary embodiments of the method for lowering HbA1c, such as specific compounds of Formula I or Ia or stereoisomers, tautomers, pharmaceutically acceptable salts, or hydrates thereof; specific daily doses of compounds of Formula I or Ia or stereoisomers, tautomers, pharmaceutically acceptable salts, or hydrates thereof; specific SGLT2 inhibitors; manner of administration of compounds of Formula I or Ia or stereoisomers, tautomers, pharmaceutically acceptable salts, or hydrates thereof and the SGLT2 inhibitor (i.e., simultaneously, sequentially, as separate compositions, or as a single composition); subject criteria, subject sub-populations; and specific diabetes-related diseases and disorders are as described in any one or more of the exemplary embodiments above.

### REFERENCES

1. Sherwani, S. I., Khan, H. A., Ekhzaimy, A., Masood, A., & Sakharkar, M. K. (2016). Significance of HbA1c Test in Diagnosis and Prognosis of Diabetic Patients. Biomarker insights, 11, 95-104.
2. World Health Organization (WHO) (2011). Use of Glycated Haemoglobin (HbA1c) in the Diagnosis of Diabetes Mellitus Abbreviated Report of a WHO Consultation. Geneva: WHO.
3. Khan, H. A., Sobki, S. H., & Khan, S. A. (2007). Association between glycaemic control and serum lipids profile in type 2 diabetic patients: HbA1c predicts dyslipidaemia. Clinical and experimental medicine, 7(1), 24-29.
4. American Diabetes Association (2011). Diagnosis and classification of diabetes mellitus. Diabetes care, 34 Suppl 1(Suppl 1), S62-S69.
5. Kharroubi, A. T., & Darwish, H. M. (2015). Diabetes mellitus: The epidemic of the century. World journal of diabetes, 6(6), 850-867.
6. International Diabetes Federation 2019. IDF Diabetes Atlas, 9th Edn. Brussels, Belgium: International Diabetes Federation.
7. Centers for Disease Control and Prevention. National Diabetes Statistics Report, 2020. Atlanta, GA: Centers for Disease Control and Prevention, U.S. Dept of Health and Human Services; 2020.
8. DeFronzo, R., A. (2004). Pathogenesis of Type 2 Diabetes Mellitus. Med Clin N Am, 88, 787-835.
9. Fowler, M. J. (2008). Microvascular and Macrovascular Complications of Diabetes. Clinical Diabetes, 26(2), 77-82.
10. Vithian, K. and Hurel, S. (2010). Microvascular complications: pathophysiology and management. Clin Med (Lond), 10(5), 505-509.
11. Beckman, J. A. and Creager, M. A. (2016). Vascular Complications of Diabetes. Circulation Research, 118, 1771-1785.
12. Klein, R., Klein, B. E., Moss, S. E., Davis, M. D., & DeMets, D. L. (1984). The Wisconsin epidemiologic study of diabetic retinopathy. III. Prevalence and risk of diabetic retinopathy when age at diagnosis is 30 or more years. Archives of ophthalmology (Chicago, Ill.: 1960), 102(4), 527-532.
13. Rangel, E. B, Rodrigues, C. O., and de Sa, J. R. (2019). Micro- and Macrovascular Complications in Diabetes Mellitus: Preclinical and Clinical Studies. J Diabetes Res, 2019, 2161085.
14. Zheng, D., Dou, J., Liu, G., Pan, Y., Yan, Y., Liu, F., Gaisano, H. Y., Lu, J., & He, Y. (2019). Association Between Triglyceride Level and Glycemic Control Among Insulin-Treated Patients With Type 2 Diabetes. The Journal of clinical endocrinology and metabolism, 104(4), 1211-1220.
15. Naqvi, S., Naveed, S., Ali, Z., Ahmad, S. M., Asadullah Khan, R., Raj, H., Shariff, S., Rupareliya, C., Zahra, F., & Khan, S. (2017). Correlation between Glycated Hemoglobin and Triglyceride Level in Type 2 Diabetes Mellitus. Cureus, 9(6), e1347.
16. Sheth, J., Shah, A., Sheth, F., Trivedi, S., Nabar, N., Shah, N., Thakor, P., & Vaidya, R. (2015). The association of dyslipidemia and obesity with glycated hemoglobin. Clinical diabetes and endocrinology, 1, 6.
17. Bae, J. P., Lage, M. J., Mo, D., Nelson, D. R., & Hoogwerf, B. J. (2016). Obesity and glycemic control in patients with diabetes mellitus: Analysis of physician electronic health records in the US from 2009-2011. Journal of diabetes and its complications, 212-220.
18. Yoon, J. W., Ha, Y. C., Kim, K. M., Moon, J. H., Choi, S. H., Lim, S., Park, Y. J., Lim, J. Y., Kim, K. W., Park, K. S., & Jang, H. C. (2016). Hyperglycemia Is Associated with Impaired Muscle Quality in Older Men with Diabetes: The Korean Longitudinal Study on Health and Aging. Diabetes & metabolism journal, 40(2), 140-146.
19. Kalyani, R. R., Metter, E. J., Egan, J., Golden, S. H., & Ferrucci, L. (2015). Hyperglycemia predicts persistently lower muscle strength with aging. Diabetes care, 38(1), 82-90.
20. Park, S. W., Goodpaster, B. H., Strotmeyer, E. S., de Rekeneire, N., Harris, T. B., Schwartz, A. V., Tylaysky, F. A., & Newman, A. B. (2006). Decreased muscle strength and quality in older adults with type 2 diabetes: the health, aging, and body composition study. Diabetes, 55(6), 1813-1818.
21. Hirata, Y., Nomura, K., Senga, Y., Okada, Y., Kobayashi, K., Okamoto, S., Minokoshi, Y., Imamura, M., Takeda, S., Hosooka, T., & Ogawa, W. (2019). Hyperglycemia induces skeletal muscle atrophy via a WWP1/KLF15 axis. JCI insight, 4(4), e124952.
22. Sugimoto, K., Tabara, Y., Ikegami, H., Takata, Y., Kamide, K., Ikezoe, T., Kiyoshige, E., Makutani, Y., Onuma, H., Gondo, Y., Ikebe, K., Ichihashi, N., Tsuboyama, T., Matsuda, F., Kohara, K., Kabayama, M., Fukuda, M., Katsuya, T., Osawa, H., Hiromine, Y., . . . Rakugi, H. (2019). Hyperglycemia in non-obese patients with type 2 diabetes is associated with low muscle mass: The Multicenter Study for Clarifying Evidence for Sarcopenia in Patients with Diabetes Mellitus. Journal of diabetes investigation, 10(6), 1471-1479.
23. Abidin Ozturk, Z. A., Turkbeyler, I. H., Demir, Z., Bilici, M., & Kepekci, Y. (2017). The effect of blood glucose regulation on sarcopenia parameters in obese and diabetic patients. Turkish journal of physical medicine and rehabilitation, 64(1), 72-79.
24. Ramirez, A., Wolfsgrubera, S., Langed, C. et al. (2015). Elevated HbA1c is Associated with Increased Risk of Incident Dementia in Primary Care Patients. J Alzheimers Dis, 44(4), 1203-12.
25. Marden, J. R., Mayeda, E. R., Tchetgen Tchetgen, E. J., Kawachi, I., and Glymour, M. M. (2017). High Hemoglobin A1c and Diabetes Predict Memory Decline in the Health and Retirement Study. Alzheimer Dis Assoc Disord, 31(1), 48-54.
26. Rawshani, A. Rawshani, A., Svensson, A.-M., and Gudbjornsdottir, S. (2015) Glycaemic control and incidence of dementia in 363,573 patients with type 2 diabetes: an observational study. Diabetologia, 58 (Suppl 1):S1-S607.
27. Celis-Morales, C., Franzen, S., Svensson, A. M., Sattar, N., and Gudbjornsdottir, S. (2020). Glycated haemoglobin, type 2 diabetes and the links to dementia and its major sub types: findings from the Swedish National Diabetes Register. Diabetologia, 63 (Suppl 1):S1-5485.
28. Stratton, I. M., Adler, A. I., Neil, H. A., Matthews, D. R., Manley, S. E., Cull, C. A., Hadden, D., Turner, R. C., & Holman, R. R. (2000). Association of glycaemia with macrovascular and microvascular complications of type 2 diabetes (UKPDS 35): prospective observational study. BMJ (Clinical research ed.), 321(7258), 405-412.
29. Khaw, K. T., Wareham, N., Bingham, S., Luben, R., Welch, A., & Day, N. (2004). Association of hemoglobin A1c with cardiovascular disease and mortality in adults: the European prospective investigation into cancer in Norfolk. Annals of internal medicine, 141(6), 413-420.
30. Selvin, E., Marinopoulos, S., Berkenblit, G., Rami, T., Brancati, F. L., Powe, N. R., & Golden, S. H. (2004). Meta-analysis: glycosylated hemoglobin and cardiovascular disease in diabetes mellitus. Annals of internal medicine, 141(6), 421-431.
31. Shimazawa, R., Ikeda, M. (2019). Imbalance in glycemic control between the treatment and placebo groups in cardiovascular outcome trials in type 2 diabetes. J of Pharm Policy and Pract 12, 30.
32. Zinman, B., Wanner, C., Lachin, J. M., et al. (2015). Empagliflozin, cardiovascular outcomes, and mortality in type 2 diabetes. N Engl J Med, 373(22), 2117-28.
33. Neal B., Perkovic V., Mahaffey, K. W., et al. (2017). Canagliflozin and cardiovascular and renal events in type 2 diabetes. N Engl J Med, 377(7), 644-657.
34. Perkovic, V., Jardine, M. J., Neal, B., et al. (2019). Canagliflozin and renal outcomes in type 2 diabetes and nephropathy. N Engl J Med, 380(24), 2295-2306.
35. Wiviott, S. D., Raz, I., Bonaca, M. P., et al. (2019). Dapagliflozin and Cardiovascular Outcomes in Type 2 Diabetes. N Engl J Med, 380(4), 347-357.
36. Owens, D. R., Monnier, L., & Barnett, A. H. (2017). Future challenges and therapeutic opportunities in type 2 diabetes: Changing the paradigm of current therapy. Diabetes, obesity & metabolism, 19(10), 1339-1352.
37. Chen, L. F., Williams, S. A., Mu, Y., Nakano, H., Duerr, J. M., Buckbinder, L., & Greene, W. C. (2005). NF-kappaB RelA phosphorylation regulates RelA acetylation. Molecular and cellular biology, 25(18), 7966-7975.
38. Villagra, A., Sotomayor, E. M., & Seto, E. (2010). Histone deacetylases and the immunological network: implications in cancer and inflammation. Oncogene, 29(2), 157-173.
39. Bayarsaihan D. (2011). Epigenetic mechanisms in inflammation. Journal of dental research, 90(1), 9-17.
40. Huang, B., Yang, X. D., Zhou, M. M., Ozato, K., & Chen, L. F. (2009). Brd4 coactivates transcriptional activation of NF-kappaB via specific binding to acetylated RelA. Molecular and cellular biology, 29(5), 1375-1387.
41. Brown, J. D., Lin, C. Y., Duan, Q., Griffin, G., Federation, A., Paranal, R. M., Bair, S., Newton, G., Lichtman, A., Kung, A., Yang, T., Wang, H., Luscinskas, F. W., Croce, K., Bradner, J. E., & Plutzky, J. (2014). NF-κB directs dynamic super enhancer formation in inflammation and atherogenesis. Molecular cell, 56(2), 219-231.
42. Das, S., Senapati, P., Chen, Z., et al. Regulation of angiotensin II actions by enhancers and super-enhancers in vascular smooth muscle cells. Nat Commun 8, 1467 (2017).
43. Ray, K. K., Nicholls, S. J., Buhr, K. A., Ginsberg, H. N., Johansson, J. O., Kalantar-Zadeh, K., Kulikowski, E., Toth, P. P., Wong, N., Sweeney, M., Schwartz, G. G., & BETonMACE investigators and Committees (2020). Effect of Apabetalone Added to Standard Therapy on Major Adverse Cardiovascular Events in Patients With Recent Acute Coronary Syndrome and Type 2 Diabetes: A Randomized Clinical Trial. JAMA, 323(16), 1565-1573.
44. Cui, J. Y., Zhou, R. R Han, S., Wang, T. S., Wang, L. Q., & Xie, X. Et (2018) Statin therapy on glycemic control in type 2 diabetic patients: A network meta-analysis. J Clin Pharm Ther. 1-15.
45. Ooba, N., Tanaka, S., Yasukawa, Y., Yoshino, N Hayashi, H., Hidaka, S., Seki, T., & Fukuoka, N. (2016) Effect of high-potency statins on HbA1c in patients with or without diabetes mellitus. Journal of Pharmaceutical Health Care and Sciences 2(8):1-6.

### EXAMPLES

### Example 1: Clinical Development

Apabetalone (RVX-208) was evaluated in a recently completed clinical Phase 3 trial (BETonMACE; NCT02586155) for the effect on MACE in type 2 diabetes patients with low HDL cholesterol (below 40 mg/dL for males and below 45 mg/dL for females) and a recent acute coronary syndrome (ACS) (preceding 7-90 days). All patients received high intensity statin treatment or maximum tolerated statin treatment, which was 20-40 mg daily or a maximum daily dose of 40 mg for rosuvastatin or 40-80 mg daily or a maximum daily dose of 80 mg for atorvastatin.

Patients (n=2425) with ACS in the preceding 7 to 90 days, with type 2 diabetes and low HDL cholesterol (≤40 mg/dl for men, ≤45 mg/dl for women), receiving intensive or maximum-tolerated therapy with atorvastatin or rosuvastatin, were assigned in double-blind fashion to receive apabetalone 100 mg orally twice daily or matching placebo. Baseline characteristics include female sex (25%), myocardial infarction as index ACS event (74%), coronary revascularization for index ACS (76%), treatment with dual anti-platelet therapy (87%) and renin-angiotensin system inhibitors (91%), median LDL cholesterol 65 mg per deciliter, and median HbA1c 7.3%. The primary efficacy measure is time to first occurrence of cardiovascular death, non-fatal myocardial infarction, or stroke. The study enrolled 2425 patients and the MACE outcome population consisted of 2418 patients.

### Example 2: Post-Hoc Analysis

In the BETonMACE clinical study, a total of N=298 patients (N=150 in RVX-208 treatment group and N=148 in placebo treatment group) were administered a SGLT2 inhibitor (empagliflozin, dapagliflozin, or canagliflozin) with specified statin therapy (atorvastatin and rosuvastatin) and other guideline-defined treatments. Specifically, a total of 150 patients received both RVX-208 and an SGLT2 inhibitor; a total of 148 received an SGLT2 inhibitor, but no RVX-208; a total of 1062 received RVX-208, but no SGLT2 inhibitor; a total of 1058 received neither RVX-208 or an SGLT2 inhibitor.

Patients who were randomized and received at least one dose of SGLT2 treatment while actively receiving study drug (RVX-208 or placebo) were counted as those receiving a combination of SGLT2 treatment with either RVX-208 or placebo. Patients receiving more than one drug therapy within the SGLT2 inhibitor class were counted only once based upon whichever drug therapy patients continued taking at the end of treatment with study drug (RVX-208 or placebo). In cases where patients were receiving more than one drug therapy within the SGLT2 inhibitor class at the end of treatment with study drug, whichever SGLT2 inhibitor therapy was received for longer was counted.

The last visit on treatment (LVT) timepoint represented the longest study exposure duration for patients receiving RVX-208 or placebo with and without SGLT2 inhibitors and is the focus of this analysis. The median time to LVT (and total study drug exposure) for patients with a baseline and LVT measurement for HbA1c for patients administered an SGLT2 inhibitor (N=298) was 744 days (2.04 years). For the patients treated with a SGLT2 inhibitor and RVX-208 (N=150), the median time to LVT was 740 days (2.03 years) and for the patients treated with a SGLT2 inhibitor and received placebo (N=148), the median time to LVT was 745 days (2.04 years). No statistical difference was observed between the duration of study drug exposure, indicating a balance was observed between treatment groups.

In patients receiving an SGLT2 inhibitor in addition to RVX-208 (N=150), the median age was 58 years, 16% were women, 92% were white, mean duration of diabetes was 9.9 years, the average BMI was 30.3 kg/m², and the baseline HbA1c was 8.2%.

In patients receiving an SGLT2 inhibitor in addition to placebo (N=148), the median age was 59 years, 18% were women, 89% were white, mean duration of diabetes was years, the average BMI was 30.2 kg/m², and the baseline HbA1c was 8.0%.

No statistical difference was observed in any of these parameters indicating a balance was observed between treatment groups.

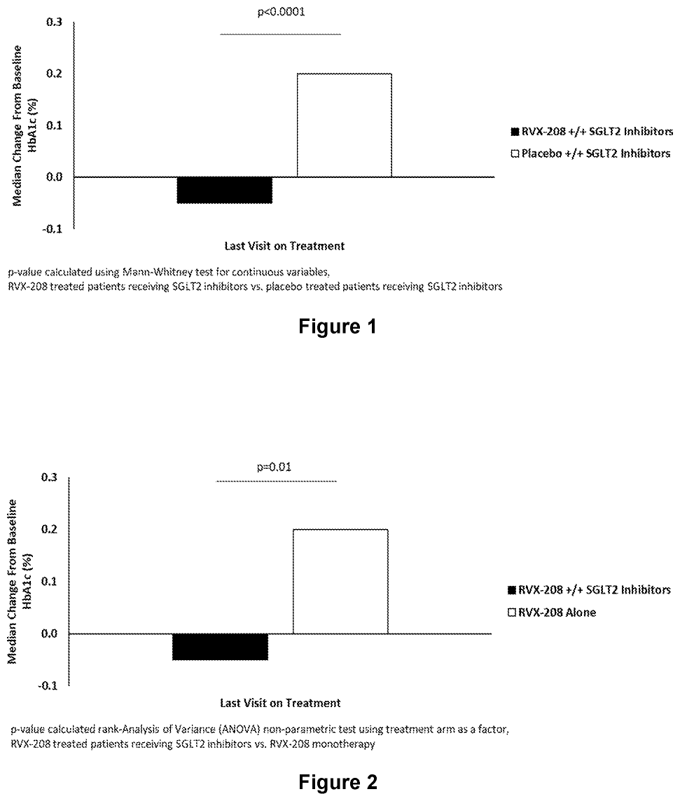

FIGS. 1-2 each compare the median change of HbA1c from baseline to LVT between two groups of patients, a test group, and a control group, which are described as follows:
i. patients treated with a SGLT2 inhibitor and RVX-208 (test) and patients treated with a SGLT2 inhibitor only and received a placebo (control) (FIG. 1); and,
ii. patients treated with RVX-208 and a SGLT2 inhibitor (test) and patients treated RVX-208 only (control) (FIG. 2).

In FIG. 1, where the patients were treated with SGLT2 inhibitors and received either RVX-208 or a placebo, the effect of the co-administration of RVX-208 and SGLT2 inhibitors—quantified using reductions in HbA1c levels from baseline—illustrated a significant reduction of HbA1c compared to placebo and SGLT2 inhibitors at LVT, with a median treatment difference of −0.25% (p<0.0001, Mann-Whitney) and mean treatment difference of −0.33% (ANOVA 95% CI, −0.09 to 0.8) (p=0.13, ANOVA; p=0.11, Rank-ANOVA).

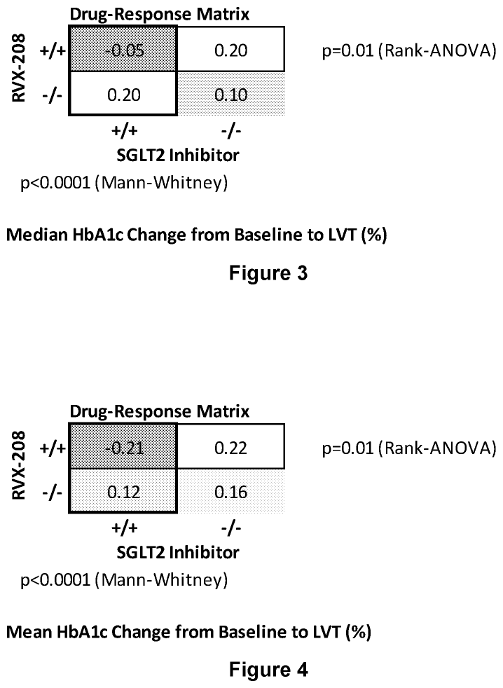

Specifically, RVX-208 in combination with SGLT2 inhibitors decreased HbA1c from a median of 8.2% at baseline to a median of 7.8% at the last visit on treatment (LVT). The mean change of HbA1c from baseline to LVT in this combination therapy group was −0.21% (FIG. 4); the median change from baseline to LVT was −0.05% (FIG. 3). Comparatively, SGLT2 inhibitor monotherapy group had a median HbA1c of 8.0% at baseline and a median HbA1c of 8.2% at LVT. The mean change from baseline to LVT in this SGLT2 inhibitor monotherapy group was +0.12% (FIG. 4); the median change from baseline to LVT was +0.20% (FIG. 3).

In FIG. 2, where patients were treated with the combination of RVX-208 and a SGLT2 inhibitor or with RVX-208 alone, the effect of the co-administration of RVX-208 and SGLT2 inhibitors—quantified using reductions in HbA1c levels from baseline—illustrated a significant reduction of HbA1c compared to RVX-208 without SGLT2 inhibitors at LVT, with a median treatment difference of −0.25% (p<0.0001, Mann-Whitney) and mean treatment difference of −0.43% (ANOVA 95% CI, 0.09 to 0.8) (p=0.01, ANOVA; p=0.01, Rank-ANOVA).

Specifically, the RVX-208 monotherapy group had a median HbA1c of 7.3% at baseline and a median HbA1c of 7.3% at LVT. The mean change of HbA1c from baseline to LVT in this RVX-208 monotherapy group was +0.22% (FIG. 4); the median change of HbA1c from baseline to LVT was +0.20% (FIG. 3). The statistical parameters for the RVX-208 and SGLT2 inhibitor combination therapy are as described above.

In conclusion, the results depicted in FIGS. 1-4 indicate that neither RVX-208 monotherapy nor SGLT2 inhibitor monotherapy was able to reduce median or mean HbA1c levels in patients with T2DM and a recent ACS. In some instances, the baseline HbA1c actually increased when measured at LVT in both RVX-208 monotherapy and SGLT2 inhibitor monotherapy. Thus, it was unexpected that a combination therapy of RVX-208 and SGLT2 inhibitor would result in any reduction in HbA1c, let alone reductions that are of statistical significance in both median and mean HbA1c changes as well as median HbA1c levels from baseline to LVT in the same patient population. Thus, the combination of a compound of Formula I with an SGLT2 inhibitor is synergistic for reducing median or mean HbA1c levels in patients with T2DM and a recent ACS.

1. A method for lowering glycated hemoglobin (HbA1c) level to treat and/or prevent a diabetes-related disease or disorder, the method comprising administering to a subject in need thereof, a combination of a sodium-glucose transport protein 2 (SGLT2) inhibitor and a compound of Formula I: 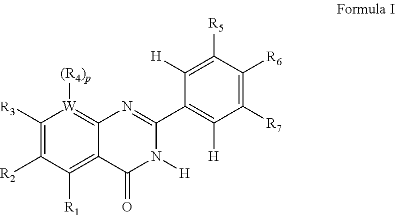
or a stereoisomer, tautomer, pharmaceutically acceptable salt, or hydrate thereof,
wherein:
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₅ and R₇ are each independently selected from alkyl, alkoxy, amino, halogen, and hydrogen;
R₆ is selected from amino, amide, alkyl, hydrogen, hydroxyl, piperazinyl, and alkoxy;
W is selected from C and N, wherein:
if W is N, then p is 0 or 1, and
if W is C, then p is 1; and
for W—(R₄)ₚ, W is C, p is 1 and R₄ is H, or W is N and p is 0.

2. The method according to claim 1, wherein the compound of Formula I is a compound of Formula Ia: 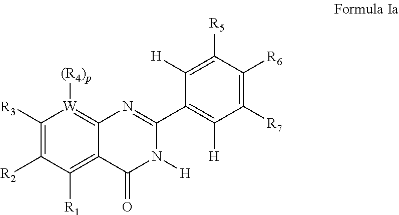
or a stereoisomer, tautomer, pharmaceutically acceptable salt, or hydrate thereof,
R₁ and R₃ are each independently selected from alkoxy, alkyl, and hydrogen;
R₂ is selected from alkoxy, alkyl, and hydrogen;
R₅ and R₇ are each independently selected from alkyl, alkoxy, amino, halogen, and hydrogen;
R₆ is selected from alkyl, hydroxyl, and alkoxy;
W is selected from C and N, wherein:
if W is N, then p is 0 or 1, and
if W is C, then p is 1; and
for W—(R₄)ₚ, W is C, p is 1 and R₄ is H, or W is N and p is 0.

3. The method according to claim 1 or claim 2, wherein the compound of Formula I or Ia is 2-(4-(2-hydroxyethoxy)-3,5-dimethylphenyl)-5,7-dimethoxyquinazolin-4(3H)-one (RVX-208 or RVX000222) or a pharmaceutically acceptable salt thereof.

4. The method according to any one of claims 1 to 3, comprising administering to the subject, a daily dose of 100-300 mg of 2-(4-(2-hydroxyethoxy)-3,5-dimethylphenyl)-5,7-dimethoxyquinazolin-4(3H)-one or an equivalent amount of a pharmaceutically acceptable salt thereof.

5. The method according to claim 5, comprising administering to the subject, a daily dose of 200 mg of 2-(4-(2-hydroxyethoxy)-3,5-dimethylphenyl)-5,7-dimethoxyquinazolin-4(3H)-one or an equivalent amount of a pharmaceutically acceptable salt thereof.

6. The method according to any one of claims 1 to 5, wherein the SGLT2 inhibitor is selected from empagliflozin, canagliflozin, dapagliflozin, bexagliflozin, ertugliflozin, sotagliflozin, luseogliflozin, tofogliflozin, and HM41322.

7. The method according to claim 6, wherein the SGLT2 inhibitor is selected from empagliflozin, canagliflozin, and dapagliflozin.

8. The method according to any one of claims 1 to 7, wherein the compound of Formula I or Ia is administered simultaneously with the SGLT2 inhibitor as separate compositions.

9. The method according to any one of claims 1 to 7, wherein the compound of Formula I is administered with the SGLT2 inhibitor as a single composition.

10. The method according to any one of claims 1 to 9, wherein the subject is a human.

11. The method according to any one of claims 1 to 10, wherein the subject is a human with receiving statin therapy.

12. The method according to any one of claims 1 to 11, wherein the subject is a human with type 2 diabetes and low HDL cholesterol (below 40 mg/dL for males and below 45 mg/dL for females) and a recent acute coronary syndrome (ACS) (preceding 7-90 days).

13. The method according to any one of claims 1 to 12, wherein the diabetes-related disease or disorder is a diabetic comorbidity associated with elevated HbA1c levels that is insulin resistance (impaired glucose homeostasis).

14. The method according to any one of claims 1 to 12, wherein the diabetes-related disease or disorder is a diabetic comorbidity associated with elevated HbA1c levels that is selected from muscle quality decline, muscle atrophy, sarcopenia, and a combination thereof.

15. The method according to any one of claims 1 to 12, wherein the diabetes-related disease or disorder is a complication of diabetes associated with elevated HbA1c levels.

16. The method according to claim 16, wherein the complication of diabetes is selected from nephropathy, neuropathy, retinopathy, and a combination thereof.

17. The method according to any one of claims 1 to 12, wherein the diabetes-related disease or disorder is a diabetic comorbidity associated with elevated HbA1c levels that is dementia associated with elevated HbA1c levels.

18. The method according to claim 17, wherein the dementia associated with elevated HbA1c levels is selected from mild cognitive impairment, vascular dementia, Alzheimer's disease dementia, Lewy body dementia, frontotemporal dementia, mixed dementia (vascular dementia and Alzheimer's disease), and a combination thereof.
